(19) United States
(12) Patent Application Publication
(10) Pub. No.: US 2007/0010410 A1
(43) Pub. Date: Jan. 11, 2007

(54) Stability profile by minimizing wall effects for a personal care composition comprising at least two phases

(76) Inventors: Michael Frederick Niebauer, Cincinnati, OH (US); Karl Shiqing Wei, Mason, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(21) Appl. No.: 11/479,766

(22) Filed: Jun. 30, 2006

Related U.S. Application Data
(60) Provisional application No. 60/696,927, filed on Jul. 6, 2005.

(51) Int. Cl. A61K 8/00; A61Q 1/14

(52) U.S. Cl. 510/130; 424/70.1

(57) ABSTRACT

A personal care product including a container having an inner wall and a personal care composition positioned within the container, the personal care composition including at least two phases, wherein only one of the two phases is in direct contact with the inner wall.

### CROSS REFERENCE TO RELATED APPLICATION

This application claims the benefit of U.S. Provisional Application No. 60/696,927 filed Jul. 6, 2005.

### FIELD

The present invention relates to personal care compositions and, more particularly, personal care compositions having at least two phases.

### BACKGROUND

Various personal care compositions have been developed that employ two or more phases packaged in a single container. The phases are, at least initially, separate and distinct. Therefore, various cleansing agents, conditioning agents, and/or benefit agents may be combined in a single package without the disadvantages typically encountered when combining various agents in a uniform or single phase composition. Multi-phase personal care compositions are discussed in greater detail in U.S. Publication No. 2004/0057920, and U.S. Publication No. 2004/0092415.

Prior art multi-phase personal care compositions have encountered several problems. One such problem is the tendency for multi-phase personal care compositions to de-stabilize, resulting in a mixing (or partial mixing) of the phases. The mixing (or partial mixing) may be a result of movement of the container or packaging holding the multi-phase personal care composition (e.g., during shipping). As the phases begin to mix, the components of one phase may react with the components of an adjacent phase. Accordingly, there is a need for a multi-phase personal care composition having improved stability.

### SUMMARY

In one aspect, the present invention is directed to a multi-phase personal care product including a container having an inner wall and a personal care composition disposed within the container, the personal care composition having at least two phases, wherein only one of the at least two phases is in direct contact with the inner wall.

In another aspect, the present invention is directed to a multi-phase personal care product including a container having a side wall and a bottom wall and a personal care composition disposed within the container, the personal care composition having at least two visually distinct phases, wherein only one of the at least two phases is in direct contact with the side wall of the container.

In another aspect, the present invention is directed to a method for packaging a personal care composition having at least two visually distinct phases. The method includes the steps of providing a container having an inner wall, introducing a first one of the at least two visually distinct phases into the container and introducing a second one of the at least two visually distinct phases into said container, wherein only the first one of the at least two visually distinct phases is in direct contact with the inner wall of the container.

Other aspects of the present invention will become apparent from the following description and the appended claims.

### DETAILED DESCRIPTION

The multi-phase personal care composition of the present invention includes at least two phases. These and other aspects of the present invention, as well as various optional ingredients suitable for use with the personal care composition, are described in greater detail herein.

The essential components of the personal care composition are described below. Also included is a nonexclusive description of various aspects and embodiments of the present invention. While the specification concludes with claims that particularly point out and distinctly claim the invention, it is believed the present invention will be better understood from the following description.

As used herein, all percentages, parts, and ratios are based upon the total weight of the composition of the present invention, unless otherwise specified. All such weights, as they pertain to listed ingredients, are based on the active level and, therefore, do not include carriers or by-products that may be included in commercially available materials, unless otherwise specified. The term “weight percent” may be denoted as “wt. %” herein. All ratios are weight ratios unless specifically stated otherwise.

Except as otherwise noted, all amounts, including quantities, percentages, portions, and proportions, are understood to be modified by the word “about,” and amounts are not intended to indicate significant digits.

As used herein, the term “visually distinct” means that the regions occupied by each phase can be separately seen by the human eye as distinctly separate regions in contact with one another (i.e., they are not emulsions or dispersions of particles of less than about 100 microns).

As used herein, the term “visibly clear” means that the transmission of the composition is greater than 60%, preferably greater than 80%. The transparency of the composition is measured using Ultra-Violet/Visible (UV/VIS) Spectrophotometry, which determines the absorption or transmission of UV/VIS light by a sample. A light wavelength of 600 nm has been shown to be adequate for characterizing the degree of clarity of personal care compositions. Typically, it is best to follow the specific instructions associated with the specific spectrophotometer being used. In general, the procedure for measuring percent transmittance starts by setting the spectrophotometer to the 600 nm. Then a calibration “blank” is run to calibrate the readout to 100 percent transmittance. The test sample is then placed in a cuvette designed to fit the specific spectrophotometer and the percent transmittance is measured by the spectrophotometer at 600 nm.

As used herein, the terms “multi-phased” or “multi-phase” mean that at least two phases occupy separate and distinct physical spaces inside the package in which they are stored, but are in direct contact with one another (i.e., they are not separated by a barrier and they are not emulsified).

As used herein, the term “stable” refers to compositions in which the visible pattern or arrangement of the phases in different locations in the package does not significantly change overtime when sitting in physical contact at ambient conditions for a period of at least about 180 days. In addition, it is meant that no separation, creaming, or sedimentation occurs.

As used herein, the term “personal care composition” refers to the compositions of the present invention, wherein the compositions are intended to include only those compositions for topical application to the hair, skin, or other regions of a human or animal body. The term “personal care composition” specifically excludes compositions that are directed to applications such as hard surface cleansing and fabric or laundry cleansing.

The present invention is directed to personal care compositions having two or more phases, wherein the phases are packaged in physical contact with each other. However, only one phase may be in physical and/or intimate contact with the inner walls, side walls, and/or the bottom wall of the packaging.

In one aspect, a personal care product may include a personal care composition packaged in a container. The container may include one or more walls, such as, for example, one or more side walls, one or more bottom walls, and/or one or more upper or top walls. Furthermore, the container may include a cap for sealing the personal care composition within the container. Without limitation, the container may be a bottle, a jug, a jar, a vessel, or the like. The container may be one in which the end from which the bottle is filled is not only the end from which the product is to be dispensed, but it is also the end upon which the bottle is intended to rest or sit upon (e.g., the bottle's base) for storage by the consumer and/or for display on the store shelf. The container may, for example, have a generally circular, obround, rectangular, square, triangular, or polygonal cross section in top view.

In one aspect, the personal care composition may include two phases. Personal care compositions having additional phases are also within the scope of the present invention. For example, the personal care composition may include three, four, or more phases packaged in a single container.

The first or inner phase may be in physical and/or intimate contact with the second or outer phase. The inner phase may be suspended or distributed within the outer phase in various patterns, such as, for example, striped, marbled, rectilinear, interrupted striped, checked, mottled, veined, clustered, speckled, geometric, spotted, ribbons, helical, swirled, arrayed, variegated, textured, grooved, ridged, waved, sinusoidal, spiraled, twisted, curved, cycle, streaked, striated, contoured, anisotropic, laced, weave or woven, basket weave, spotted and tessellated.

According to one aspect of the present invention, the rheology and chemical composition of the phases may be selected and/or adjusted to improve stability (i.e., the ability of a phase to remain separate and/or distinct from an adjacent phase). For example, the phase stability may be improved by adjusting the viscosity and/or composition such that materials from one phase do not enter, combine or mix with materials from adjacent phases. In one aspect, the viscosity may be adjusted (e.g., using thickeners) such that one or more phases has a high viscosity (e.g., about 15 to about 20,000, about 25 to about 10,000, about 40 to about 5,000, and/or about 50 to about 2,000 Pas) at zero or low shear rates (e.g., about 0.0005 to about 0.005 s⁻¹) and a shear thinning profile that goes to a low viscosity (e.g., about 0 to about 50, about 0 to about 20, and/or about 1 to about 15 Pas) at high or higher shear rates (e.g., about 10 to about 1000 s⁻¹).

In one aspect, the stability of the phases may be improved or enhanced by filling the container with the multi-phase personal care composition such that the inner phase is in contact with the bottom wall of the container, but not in contact with the side walls of the container. Alternatively, only one phase of the composition may be in intimate or direct contact with any portion of the inner walls of the container (i.e., the inner phase is not in contact with either the side or bottom walls of the container).

Accordingly, a gap may be formed between the side and/or bottom walls of the container and the inner phase. The gap may have various lengths throughout the product. In one aspect, the gap may be from about 0.1 to about 49 percent of the diameter, length, width or depth of the container. The gap may also be from about 0.2 to about 40 percent of the diameter, length, width, or depth of the container. In another aspect, the gap may be about 5 to about 25 percent of the diameter, length, width, or depth of the container.

At this point it should be understood that the gap may vary throughout the container. Alternatively, the container may be filled with the composition such that the gap throughout the container is substantially consistent or equal.

Without being limited to any particular theory, it is believed that by allowing only one phase to contact the side and/or bottom walls of the container (i.e., providing a gap), the fluid dynamic forces acting at the inner walls of the container during, for example, transportation of the container only affect the phase directly contacting the wall. Specifically, the velocity profile at the inner wall of the container is zero and increases to the center of the container. Therefore, stability is maintained and phase mixing is avoided when only one phase is in the zero-velocity region of the container.

The two or more phases may be physically and visually distinct. In one aspect, the visually distinct phases may be of a different color, opacity or refractive index, thereby allowing the human eye to distinguish between the phases. In another aspect, one phase (or phases) may be visibly clear and the other phase (or phases) may be opaque or visibly not clear. In another aspect, the outer (i.e., the phase contacting the inner wall of the container) may be clear such that the inner phase (or phases) may be seen through the outer phase.

The phases may be made visually distinct using dyes, pigments, pearlescent agents, lakes, colorants, or mixtures thereof. Colorants useful according to the present invention may include, for example, Red 30 Low Iron, FD&C Red 40 AL Lake, D&C Red Lake Blend of Lake 27 & Lake 30, FD&C Yellow 5 Al Lake, FD&C Yellow 6 Al Lake, FD&C Yellow 5 Lake, FD&C Blue #1 AL Lake, Kowet Titanium Dioxide, D&C Red 30 Talc Lake, D&C Red 6 Barium Lake, D&C Red 7 Calcium Lake, D&C Red 34 Calcium Lake, D&C Red 30 AL lake, D&C Red 27 AL lake, D&C Yellow 10 AL lake, D&C Red 21 AL Lake, Yellow Iron Oxide, D&C Red 30 Lake, Octocir Yellow 6 AL Lake, Octocir Yellow 5 AL Lake, D&C Red 28 Lake, D&C Orange 5 Zirc Al Lake, Cos Red Oxide BC, Cos Iron Oxide Red BC, Cos Iron oxide Black BC, Cos Iron Oxide Yellow, Cos Iron Oxide Brown, Cos Iron Oxide Yellow BC, Euroxide Red Unsteril, Euroxide Black Unsteril, Euroxide Yellow Steril, Euroxide Black Steril, Euroxide Red, Euroxide Black, Hydrophobic Euroxide Black, Hydrophobic Euroxide Yellow, Hydrophobic Euroxide Red, D&C Yellow 6 Lake, D&C Yellow 5 Zr Lake and mixtures thereof.

The personal care compositions of the present invention may include, but are not limited to, shampoo, conditioner, antiperspirant, deodorant, hair styling products, cleansers, soaps, bar soap, bodywash, cosmetics, foundations, lotions, creams, ointments, and hydro-alcoholic solutions. The phases may be one or a combination of a cleansing phase, a conditioning phase, a benefit phase, or the like. Further, one or more of the phases may also comprise additional components such as conditioning agents such as hydrolysed collagen with tradename Peptein 2000 available from Hormel, water soluble and water insoluble vitamins such as vitamin A, D, B1, B2, B6, B12, C, biotin, vitamin E with tradename Emix-d available from Eisai, panthenol available from Roche, pantothenic acid, panthenyl ethyl ether available from Roche, and their derivatives; hydrolysed keratin, proteins, plant extracts, and nutrients; emollients such as PPG-3 myristyl ether with tradename Varonic APM available from Goldschmidt, Trimethyl pentanol hydroxyethyl ether, PPG-11 stearyl ether with tradename Varonic APS available from Goldschmidt, Stearyl heptanoate with tradename Tegosoft SH available from Goldschmidt, Lactil (mixture of Sodium lactate, Sodium PCA, Glycine, Fructose, Urea, Niacinamide, Glucosamine, Inositol, Sodium Benzoate, and Lactic acid) available from Goldschmidt, Sodium lactate, Sodium PCA, Glycine, Fructose, Urea, Niacinamide, Glucosamine, Inositol, Sodium Benzoate, Lactic acid, Ethyl hexyl palmitate with tradename Saracos available from Nishin Seiyu and with tradename Tegosoft OP available from Goldschmidt; hair-fixative polymers such as amphoteric fixative polymers, cationic fixative polymers, anionic fixative polymers, nonionic fixative polymers, and silicone grafted copolymers; preservatives such as benzyl alcohol, methyl paraben, propyl paraben and imidazolidinyl urea; pH adjusting a gents, such as citric acid, sodium citrate, succinic acid, phosphoric acid, sodium hydroxide, sodium carbonate; salts, in general, such as potassium acetate and sodium chloride; coloring agents, such as any of the FD&C or D&C dyes, oxidative dyes and interference pigments; hair oxidizing (bleaching) agents, such as hydrogen peroxide, perborate and persulfate salts, carbonate; hair reducing agents such as the thioglycolates; perfumes; and sequestering agents, such as disodium ethylenediamine tetra-acetate; ultraviolet and infrared screening and absorbing agents such as octyl salicylate; antimicrobial agents; suspending agents; viscosity modifiers; nonvolatile solvents or diluents (water soluble and insoluble), pearlescent aids, foam boosters, additional surfactants or nonionic cosurfactants, pediculocides, chelants, skin active agents, sunscreens, UV absorbers, and, water soluble and insoluble amino acids such as asparagine, alanin, indole, glutamic acid, tyrosine, tryptamine, and their salts; and antidandruff agents such as zinc pyrithione, pyridinethione salts, azoles, climbazole, octopirox, salicylic acid, selenium sulfide, particulate sulfur and mixtures thereof.

### EXAMPLES 1-10

The compositions illustrated in the following examples exemplify specific embodiments of the compositions of the present invention. However, they are not intended to limit the present invention in any way.

The compositions illustrated in the following examples are prepared by conventional formulation and mixing methods. All exemplified amounts are listed as weight percents and exclude minor materials such as diluents, preservatives, color solutions, imagery or conceptual ingredients, botanicals and so forth, unless otherwise specified.

Personal care compositions were prepared according to the present invention. Each sample composition includes an outer phase (i.e., phase A), and an inner phase (i.e., phase B). The two phases were prepared separately. The ratios of the first phase to the second phase in the final compositions (i.e., the ratios of phase A to phase B) are provided below.

### Examples 1-10

### Shampoo Compositions

TABLE 1

| | Sample - Phase A | | | | | | | | | |
|---|---|---|---|---|---|---|---|---|---|---|
| | 1 | 2 | 3 | 4 | 5 | 6 | 7 | 8 | 9 | 10 |
| Ammonium Laureth-3 Sulfate | 10 | | | 10 | | | 6 | 6 | 8 | |
| Ammonium Lauryl Sulfate | 6 | | | 4 | | | 10 | 10 | 10 | |
| Sodium Laureth-3 Sulfate | | 8 | 6 | | 6 | 10 | | | | 10 |
| Sodium Lauryl Sulfate | | 8 | 10 | | 10 | 6 | | | | 7 |
| Cocamidopropyl Betaine FB | | | | 1 | | | | | | |
| Sodium Lauraoamphoacetate | | | | 1 | | | | | | |
| Cocamide MEA | 0.80 | 0.80 | 1.50 | 0.80 | 0.80 | 0.80 | 1.35 | 1.35 | 0.80 | 0.80 |
| Ethylene Glycol Distearate | 0 | 0 | 0 | 0 | 0 | 0 | 0 | 0 | 0 | 0 |
| Cetyl Alcohol | 0.6 | | 0.9 | | | | | | | 0.45 |
| Polyquat 10 | 0.1 | | 0.1 | | 0.25 | | | 0.5 | 0.05 | 0.1 |
| Guar Hydroxypropyl trimonium Chloride | | 0.25 | | 0.05 | | | 0.2 | | | |
| Polymethacrylamidopropyl trimonium Chloride | | | | | | 0.4 | | | 0.2 | |
| Carbopol Aqua SF-1 | 1.5 | 3 | 2.5 | 2 | 2 | 2.75 | 1.75 | 1.5 | 1.5 | 2 |
| PEG 14M | 0.1 | | 0.05 | 0.15 | | 0.05 | 0.1 | 0.05 | 0.1 | 0.1 |
| Dimethicone | 0 | 0 | 0 | 0 | 0 | 0 | 0 | 0 | 0 | 0 |
| Perfume Solution | 0.8 | 0.8 | 0.8 | 0.8 | 0.8 | 0.8 | 0.8 | 0.8 | 0.8 | 0.8 |
| Citric Acid | 0.4 | 0.9 | 0.8 | 0.8 | 0.8 | 0.8 | 0.4 | 0.4 | 0.4 | 0.8 |
| Sodium Hydroxide | 0.35 | 0.5 | 0.4 | 0.35 | 0.35 | 0.5 | 0.35 | 0.5 | 0.35 | 0.35 |
| Ammonium Xylene Sulfonate | | | | | | | | 1 | | |
| Sodium Xylene Sulfonate | | 1.5 | 0.5 | | | 0.75 | | | | |

TABLE 2

| | Sample - Phase B | | | | | | | | | |
|---|---|---|---|---|---|---|---|---|---|---|
| | 1 | 2 | 3 | 4 | 5 | 6 | 7 | 8 | 9 | 10 |
| Ammonium Laureth-3 Sulfate | 10 | | | 10 | | | 6 | 6 | 8 | |
| Ammonium Lauryl Sulfate | 6 | | | 4 | | | 10 | 10 | 10 | |
| Sodium Laureth-3 Sulfate | | 8 | 6 | | 6 | 10 | | | | 10 |
| Sodium Lauryl Sulfate | | 8 | 10 | | 10 | 6 | | | | 7 |
| Cocamidopropyl Betaine FB | | | | 1 | | | | | | |
| Sodium Lauraoamphoacetate | | | | 1 | | | | | | |
| Cocamide MEA | 0.80 | 0.80 | 1.50 | 0.80 | 0.80 | 0.80 | 1.35 | 1.35 | 0.80 | 0.80 |
| Ethylene Glycol Distearate | 1.00 | 1.50 | 1.50 | 1.50 | 1.50 | 1.00 | 1.50 | 1.50 | 1.50 | 1.25 |
| Cetyl Alcohol | 0.6 | | 0.9 | | | | | | | 0.45 |
| Polyquat 10 | 0.1 | | 0.1 | | 0.25 | | | 0.5 | 0.05 | 0.1 |
| Guar Hydroxypropyl trimonium Chloride | | 0.25 | | 0.05 | | | 0.2 | | | |
| Polymethacrylamidopropyl trimonium Chloride | | | | | | 0.4 | | | 0.2 | |
| Carbopol Aqua SF-1 | 1.5 | 3 | 2.5 | 2 | 2 | 2.75 | 1.75 | 1.5 | 1.5 | 2 |
| PEG 14M | 0.1 | | 0.05 | 0.15 | | 0.05 | 0.1 | 0.05 | 0.1 | 0.1 |
| Dimethicone | 3 | 3 | 3 | 1.2 | 3 | 0 | 3.75 | 2.5 | 7 | 5 |
| Perfume Solution | 0.8 | 0.8 | 0.8 | 0.8 | 0.8 | 0.8 | 0.8 | 0.8 | 0.8 | 0.8 |
| Citric Acid | 0.4 | 0.9 | 0.8 | 0.8 | 0.8 | 0.8 | 0.4 | 0.4 | 0.4 | 0.8 |
| Sodium Hydroxide | 0.35 | 0.5 | 0.4 | 0.35 | 0.35 | 0.5 | 0.35 | 0.5 | 0.35 | 0.35 |
| Ammonium Xylene Sulfonate | | | | | | | | 1 | | |
| Sodium Xylene Sulfonate | | 1.5 | 0.5 | | | 0.75 | | 0 | | |
| Ratio Phase A/Phase B | 70:30 | 67:33 | 50:50 | 10:90 | 33:67 | 90:10 | 67:33 | 60:40 | 90:10 | 60:40 |

### Examples 11-20

### Conditioner Compositions

| | | | | | | | | | |
|---|---|---|---|---|---|---|---|---|---|
| 11 | 12 | 13 | 14 | 15 | 16 | 17 | 18 | 19 | 20 |

Phase A Composition

| | | | | | | | | | | |
|---|---|---|---|---|---|---|---|---|---|---|
| Acrylates/aminoacrylates/C10-30 | 3 | 2.5 | 3 | 2.5 | 3 | 2.5 | 3 | 2.5 | 3 | 2.5 |
| alkyl PEG-20 itaconate copolymer | | | | | | | | | | |
| (Structure Plus, National Starch) | | | | | | | | | | |
| Hydroxypropyl guar (Jaguar HP- | 1 | 1 | 1 | 1 | 1 | 1 | 1 | 1 | 1 | 1 |
| 105, Rhodia) | | | | | | | | | | |
| L-Glutamic acid | 0.577 | | 0.577 | | 0.577 | | 0.577 | | 0.577 | |
| Hydrochloric acid | | 0.15 | | 0.15 | | 0.15 | | 0.15 | | 0.15 |
| Disodium EDTA | 0.127 | 0.127 | 0.127 | 0.127 | 0.127 | 0.127 | 0.127 | 0.127 | 0.127 | 0.127 |
| Benzyl alcohol | 0.4 | 0.4 | 0.4 | 0.4 | 0.4 | 0.4 | 0.4 | 0.4 | 0.4 | 0.4 |
| Kathon CG | 0.033 | 0.033 | 0.033 | 0.033 | 0.033 | 0.033 | 0.033 | 0.033 | 0.033 | 0.033 |
| Water and Minors (QS to 100%) | | | | | | | | | | |

Phase B Composition

| | | | | | | | | | | |
|---|---|---|---|---|---|---|---|---|---|---|
| BTMAC | 3.381 | 3.381 | 3.381 | 3.381 | 3.381 | 3.381 | | | | |
| BAPDMA | | | | | | | 2.3 | 2.3 | 2.3 | 2.3 |
| Isopropanol | 0.899 | 0.899 | 0.899 | 0.899 | 0.899 | 0.899 | | | | |
| L-Glutamic acid | | | | | | | 0.64 | 0.64 | 0.64 | 0.64 |
| Cetyl alcohol | 2.32 | 2.32 | 2.32 | 2.32 | 2.32 | 2.32 | 2.5 | 2.5 | 2.5 | 2.5 |
| Stearyl alcohol | 4.18 | 4.18 | 4.18 | 4.18 | 4.18 | 4.18 | 4.5 | 4.5 | 4.5 | 4.5 |
| 18 MM/200 cSt dimethicone (15/85 | 10 | 10 | 8 | 8 | 4 | 4 | | | | |
| blend) | | | | | | | | | | |
| Dimethyicone/cyclomethicone | | | | | | | 21 | 21 | 8.2 | 8.2 |
| (15/85 blend) | | | | | | | | | | |
| Disodium EDTA | 0.127 | 0.127 | 0.127 | 0.127 | 0.127 | 0.127 | | | | |
| EDTA | | | | | | | 0.1 | 0.1 | 0.1 | 0.1 |
| Benzyl alcohol | 0.4 | 0.4 | 0.4 | 0.4 | 0.4 | 0.4 | 0.4 | 0.4 | 0.4 | 0.4 |
| Kathon CG | 0.033 | 0.033 | 0.033 | 0.033 | 0.033 | 0.033 | 0.033 | 0.033 | 0.033 | 0.033 |
| Perfume | 0.35 | 0.35 | 0.35 | 0.35 | 0.5 | 0.5 | 0.3 | 0.3 | 0.3 | 0.3 |
| dl-pantyl | 0.05 | 0.05 | 0.05 | 0.05 | | | 0.05 | 0.05 | 0.05 | 0.05 |
| dl-panthenol | 0.05 | 0.05 | 0.05 | 0.05 | | | 0.05 | 0.05 | 0.05 | 0.05 |
| sodium hydroxide | 0.014 | 0.014 | 0.014 | 0.014 | 0.014 | 0.014 | | | | |
| Water and Minors (QS to 100%) | | | | | | | | | | |
| Ratio of Phase A to Phase B | 80/20 | 80/20 | 75/25 | 75/25 | 50/50 | 50/50 | 80/20 | 80/20 | 50/50 | 50/50 |

### Examples 21-30

### Conditioner Examples

| | | | | | | | | | |
|---|---|---|---|---|---|---|---|---|---|
| 21 | 22 | 23 | 24 | 25 | 26 | 27 | 28 | 29 | 30 |

Phase A Composition

| | | | | | | | | | | |
|---|---|---|---|---|---|---|---|---|---|---|
| Ammonium Laureth-3 Sulfate | 10 | | | 10 | | | 6 | 6 | 8 | |
| Ammonium Lauryl Sulfate | 6 | | | 4 | | | 10 | 10 | 10 | |
| Sodium Laureth-3 Sulfate | | 8 | 6 | | 6 | 10 | | | | 10 |
| Sodium Lauryl Sulfate | | 8 | 10 | | 10 | 6 | | | | 7 |
| Cocamidopropyl Betaine FB | | | | 1 | | | | | | |
| Sodium Lauraoamphoacetate | | | | 1 | | | | | | |
| Cocamide MEA | 0.80 | 0.80 | 1.50 | 0.80 | 0.80 | 0.80 | 1.35 | 1.35 | 0.80 | 0.80 |
| Ethylene Glycol Distearate | 0 | 0 | 0 | 0 | 0 | 0 | 0 | 0 | 0 | 0 |
| Cetyl Alcohol | 0.6 | | 0.9 | | | | | | | 0.45 |
| Polyquat 10 (1) | 0.1 | | 0.1 | | 0.25 | | | 0.5 | 0.05 | 0.1 |
| Guar Hydroxypropyl trimonium | | 0.25 | | 0.05 | | | 0.2 | | | |
| Chloride (2) | | | | | | | | | | |
| Polymethacrylamidopropyl | | | | | | 0.4 | | | 0.2 | |
| trimonium Chloride (3) | | | | | | | | | | |
| Carbopol Aqua SF-1 (4) | 1.5 | 3 | 2.5 | 2 | 2 | 2.75 | 1.75 | 1.5 | 1.5 | 2 |
| PEG 14M (5) | 0.1 | | 0.05 | 0.15 | | 0.05 | 0.1 | 0.05 | 0.1 | 0.1 |
| Dimethicone (6) | 0 | 0 | 0 | 0 | 0 | 0 | 0 | 0 | 0 | 0 |
| Perfume Solution | 0.8 | 0.8 | 0.8 | 0.8 | 0.8 | 0.8 | 0.8 | 0.8 | 0.8 | 0.8 |
| Citric Acid | 0.4 | 0.9 | 0.8 | 0.8 | 0.8 | 0.8 | 0.4 | 0.4 | 0.4 | 0.8 |
| Sodium Hydroxide | 0.35 | 0.5 | 0.4 | 0.35 | 0.35 | 0.5 | 0.35 | 0.5 | 0.35 | 0.35 |
| Ammonium Xylene Sulfonate | | | | | | | | 1 | | |
| Sodium Xylene Sulfonate | | 1.5 | 0.5 | | | 0.75 | | | | |
| Water and Minors (QS to 100%) | | | | | | | | | | |

Phase B Composition

| | | | | | | | | | | |
|---|---|---|---|---|---|---|---|---|---|---|
| Ammonium Laureth-3 Sulfate | 10 | | | 10 | | | 6 | 6 | 8 | |
| Ammonium Lauryl Sulfate | 6 | | | 4 | | | 10 | 10 | 10 | |
| Sodium Laureth-3 Sulfate | | 8 | 6 | | 6 | 10 | | | | 10 |
| Sodium Lauryl Sulfate | | 8 | 10 | | 10 | 6 | | | | 7 |
| Cocamidopropyl Betaine FB | | | | 1 | | | | | | |
| Sodium Lauraoamphoacetate | | | | 1 | | | | | | |
| Cocamide MEA | 0.80 | 0.80 | 1.50 | 0.80 | 0.80 | 0.80 | 1.35 | 1.35 | 0.80 | 0.80 |
| Ethylene Glycol Distearate | 1.00 | 1.50 | 1.50 | 1.50 | 1.50 | 1.00 | 1.50 | 1.50 | 1.50 | 1.25 |
| Cetyl Alcohol | 0.6 | | 0.9 | | | | | | | 0.45 |
| Polyquat 10 (1) | 0.1 | | 0.1 | | 0.25 | | | 0.5 | 0.05 | 0.1 |
| Guar Hydroxypropyl trimonium | | 0.25 | | 0.05 | | | 0.2 | | | |
| Chloride (2) | | | | | | | | | | |
| Polymethacrylamidopropyl | | | | | | 0.4 | | | 0.2 | |
| trimonium Chloride (3) | | | | | | | | | | |
| Carbopol Aqua SF-1 (4) | 1.5 | 3 | 2.5 | 2 | 2 | 2.75 | 1.75 | 1.5 | 1.5 | 2 |
| PEG 14M (5) | 0.1 | | 0.05 | 0.15 | | 0.05 | 0.1 | 0.05 | 0.1 | 0.1 |
| Dimethicone (6) | 3 | 3 | 3 | 1.2 | 3 | 0 | 3.75 | 2.5 | 7 | 5 |
| Perfume Solution | 0.8 | 0.8 | 0.8 | 0.8 | 0.8 | 0.8 | 0.8 | 0.8 | 0.8 | 0.8 |
| Citric Acid | 0.4 | 0.9 | 0.8 | 0.8 | 0.8 | 0.8 | 0.4 | 0.4 | 0.4 | 0.8 |
| Sodium Hydroxide | 0.35 | 0.5 | 0.4 | 0.35 | 0.35 | 0.5 | 0.35 | 0.5 | 0.35 | 0.35 |
| Ammonium Xylene Sulfonate | | | | | | | | 1 | | |
| Sodium Xylene Sulfonate | | 1.5 | 0.5 | | | 0.75 | | 0 | | |
| Water and Minors (QS to 100%) | | | | | | | | | | |
| Ratio of Phase A to Phase B | 70:30 | 67:33 | 50:50 | 10:90 | 33:67 | 90:10 | 67:33 | 60:40 | 90:10 | 60:40 |

(1) Polymer LR30M available from Amerchol/Dow Chemical
(2) Jaguar C17 available from Rhodia
(3) Polycare 133 available from Rhodia
(4) Carbopol Aqua SF-1 available from Noveon
(5) PEG14M, WSR N-3000 available from Union Carbide/Dow Chemical
(6) Viscasil 330M available from General Electric Silicones

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

All documents cited in the Background, Summary of the Invention, and Detailed Description of the Invention are, in relevant part, incorporated herein by reference; the citation of any document is not to be construed as an admission that it is prior art with respect to the present invention.

What is claimed is:

1. A multi-phase personal care product comprising:
a container having an inner wall; and
a personal care composition disposed within said container, said personal care composition including at least two visually distinct phases, wherein only one of said at least two phases is in direct contact with said inner wall;
wherein said inner wall and the phase not in direct contact with said inner wall define a gap therebetween.

2. The multi-phase personal care product of claim 1 wherein said phase in direct contact with said inner wall is positioned within said gap.

3. The multi-phase personal care product of claim 1 wherein said gap is from about 0.1 to about 50 percent of at least one of a diameter, a length, a width, and a depth of said container.

4. The multi-phase personal care product of claim 1 wherein at least one of said at least two phases has a high viscosity at a low shear rate and a shear thinning profile that goes to a low viscosity at a high shear rate.

5. The multi-phase personal care product of claim 4 wherein said high viscosity is from about 15 Pas to about 20,000 Pa-s at a said low shear rate of from about 0.0005 s⁻¹ to about 0.005 s⁻¹.

6. The multi-phase personal care product of claim 4 wherein said low viscosity is from about 0 Pas to about 50 Pas at a said high shear rate of from about 10 s⁻¹ to about 1000 s⁻¹.

7. The multi-phase personal care product of claim 4 wherein said high viscosity is from about 15 Pas to about 20,000 Pas at a said low shear rate of from about 0.0005 s⁻¹ to about 0.005 s⁻¹ and said low viscosity is from about 0 Pas to about 50 Pas at a said high shear rate of from about 10 s⁻¹ to about 1000 s⁻¹.

8. The multi-phase personal care product of claim 4 wherein said high viscosity is from about 25 Pas to about 10,000 Pas at a said low shear rate of from about 0.0005 s⁻¹ to about 0.005 s⁻¹.

9. The multi-phase personal care product of claim 4 wherein said low viscosity is from about 0 Pas to about 20 Pas at a said high shear rate of from about 10 s⁻¹ to about 1000 s⁻¹.

10. The multi-phase personal care product of claim 4 wherein said high viscosity is from about 25 Pas to about 10,000 Pas at a said low shear rate of from about 0.0005 s⁻¹ to about 0.005 s⁻¹ and said low viscosity is from about 0 Pas to about 20 Pas at a said high shear rate of from about 10 s⁻¹ to about 1000 s⁻¹.

11. The multi-phase personal care product of claim 1 wherein at least one of a color, an opacity and a refractive index of said phase in direct contact with said inner wall is unique with respect to said phase not in direct contact with said inner wall.

12. The multi-phase personal care product of claim 1 wherein said phase in direct contact with said inner wall is visibly clear.

13. The multi-phase personal care product of claim 12 wherein said phase not in direct contact with said inner wall includes at least one of a dye, a pigment, a pearlescent agent, a lake, and a colorant.

14. The multi-phase personal care product of claim 1 wherein said container has a side wall and a bottom wall.

15. The multi-phase personal care product of claim 1 wherein said phase not in direct contact with said inner wall comprises an additional component.

16. A method for packaging a personal care composition having at least two visually distinct phases comprising the steps of:
providing a container having an inner wall;
introducing a first one of said at least two visually distinct phases into said container; and
introducing a second one of said at least two visually distinct phases into said container,
wherein only said first one of said at least two visually distinct phases is in direct contact with said inner wall of said container.

17. The method of claim 16 wherein said first one of said at least two visually distinct phases is visibly clear.
